(19) United States
(12) Patent Application Publication
(10) Pub. No.: US 2014/0066850 A1
(43) Pub. Date: Mar. 6, 2014

(54) INFUSION PUMP PRESSURE PLATE ADAPTER SYSTEM

(71) Applicants: Renee Robert, St. Paul, MN (US); Steve Cote, Stillwater, MN (US); Jeff Taylor, Forest Lake, MN (US)

(72) Inventors: Renee Robert, St. Paul, MN (US); Steve Cote, Stillwater, MN (US); Jeff Taylor, Forest Lake, MN (US)

(21) Appl. No.: 13/602,836

(22) Filed: Sep. 4, 2012

(51) Int. Cl. A61M 5/14; A61M 39/10

(52) U.S. Cl. CPC A61M 5/1413; A61M 39/1011
USPC 604/151; 604/533; 604/262; 604/244; 29/888.02

(57) ABSTRACT

A medical infusion pump pressure plate adapter system and components that include a tube set, a control module and a reusable adapter plate in various embodiments. The tube set having a fluid tube for supplying fluid, medication, or nutrients, and an interface card component having a generally flat top surface portion fixed to the exterior surface of the fluid tube at an intermediate location of the fluid tube. The control module having a processor and a chassis with a pumping mechanism for pumping fluid through the fluid tube. Further, the reusable adapter plate has a first end pivotally mounted to the chassis of the control module for long-term pivotal retention and a second end selectively mounted to chassis of the control panel. The reusable adapter plate contains a recessed feature configured for receipt of the interface card component of the tube set.

### FIELD OF THE INVENTION

The present invention relates to an adapter system for medical infusion pumps and various apparatus and methods thereof. More specifically, this invention relates to a reusable pressure plate adapter system that couples a reusable pressure plate adapter with the pump chassis of an infusion pump and advantageous disposable tube sets.

### BACKGROUND OF THE INVENTION

Infusion pumps and medication delivery devices are well-known and widely used throughout the world today. Intravenous infusion pumps were developed decades ago to pump fluid medicants or nutrients through tubing to a patient in connection with treatment of a medical condition. Infusion pumps include peristaltic pumps, roller pumps, or expulsor pumps, for example. Many of these infusion pumps operate by governing the movement of fluid through a delivery tube by selectively occluding portions of the tube by depressing a valve or mechanism against the tube. For example, in some designs the mechanism of a pump selectively engages the tube against the pressure plate in a peristaltic fashion to force fluid through the tube.

Various infusion pumps deliver medication from either a cassette type of tube arrangement or from an administration type of tube arrangement. Accordingly, some common pumps include a reusable control module detachably coupled to a pressure plate top surface of a disposable fluid reservoir cassette. Fluid is pumped from the cassette by the reusable control module when the cassette is coupled to the control module. Alternatively, other variations of infusion pumps include reusable pump control modules and administration-type tube arrangements that utilize remote fluid reservoirs. In these arrangements, fluid is pumped from the remote fluid reservoir by the reusable control module when the administration tube arrangement is coupled to the control module.

Improvements to these types of infusion pump arrangements are desired. For example, use of cassettes can result in a fair amount of environmental waste and cost as the cassettes are generally disposable and must be frequently replaced. Further, filling sealed disposable cassettes and eliminating air bubbles, etc. from these cassettes can be time consuming and cause some difficulties to persons like pharmacy technicians responsible for filling these components. Ensuring tube retention and alignment is a necessity to the production of effective infusion pumps. Due to the importance of delivery accuracy and precision in both administration-type arrangements and cassette-type arrangements, any improvements in these areas would be appreciated and a design which could better optimize the interaction of the pump and pressure plate type features would be welcome.

Therefore, improved methods and apparatus for the chassis, pressure plate, and fluid delivery arrangement of an infusion pump are desired.

### SUMMARY OF THE INVENTION

The present invention overcomes the problems of the prior art by providing a method and device for adapting an infusion pump to operate with reduced disposable components in a design providing an enhanced arrangement of improved alignment and retention of tube components with increased accuracy and effectiveness.

One embodiment of the present invention is directed to a medical infusion pump pressure plate adapter system that includes a tube set, a control module and a reusable adapter plate. The tube set includes a fluid tube for supplying fluid, medication, or nutrients, and an interface card component having a generally flat top surface portion fixed to the exterior surface of the fluid tube at an intermediate location of the fluid tube. The control module generally includes a processor and a chassis with a pumping mechanism for pumping fluid through the fluid tube. Further, the reusable adapter plate has a first end pivotally mounted to the chassis of the control module for long-term pivotal retention and a second end selectively mounted to chassis of the control module. The reusable adapter plate contains a recessed feature configured for receipt of the interface card component of the tube set.

Another embodiment of the present invention is directed to a disposable tube set for a medical infusion pump. The disposable tube set includes a fluid tube for supplying fluids, medication, or nutrients, as well as an interface card component with a generally flat top surface portion coupled to the exterior surface of the fluid tube at an intermediate location of the fluid tube. The interface card component is configured to releasably couple to a recessed feature of a reusable adapter plate of an infusion pump.

A further embodiment of the invention is directed to a reusable adapter plate for use with a control module of an infusion pump. The reusable adapter plate includes an elongate body having a first end with a pivotal mounting structure with one or more apertures for long-term pivotal infusion pump attachment to the control module, a second end including a pump securing extension for selective engagement with the control module, and a top portion configured to retain an interface card component that is coupled to a fluid tube between the first end and the second end.

According to an embodiment of the present invention, a method for a medical infusion pump pressure plate adapter system is provided. The method includes providing a disposable tube set adapted for attachment to a reusable adapter plate having a reusable cassette reservoir housing. The tube set includes a fluid tube with a bonded interface card component and a fluid bag. The method further includes providing instructions to fill the bag of fluid, placing the bag of fluid into an interior portion of the cassette reservoir housing of the infusion pump, coupling an interface card component within a recess of the reusable adapter plate and aligning the fluid tube between a pair of positive stop components, and securing the reusable adapter plate and the cassette reservoir housing together.

### BRIEF DESCRIPTION OF THE DRAWINGS

The invention may be more completely understood in consideration of the following detailed description of various embodiments of the invention in connection with the accompanying drawings, in which:

FIG. 1A is a fragmentary, unassembled perspective view of a pump control module, a tube, pressure plate and a cassette of an infusion pump system of the prior art.

FIG. 1B is a fragmentary, unassembled perspective view of a pump control module, a tube, an alternative pressure plate and administration components of an infusion pump system of the prior art.

FIG. 2 is an unassembled perspective view of an infusion pump system which uses a reusable cassette arrangement with a disposable tube set, according to an embodiment of the invention.

FIG. 3 is a bottom view of the chassis of an infusion pump, according to an embodiment of the invention.

FIG. 4A is an unassembled, perspective view of a reusable pressure plate adapter and cassette for an infusion pump, according to an embodiment of the invention.

FIG. 4B is an unassembled, perspective view of a reusable pressure plate adapter with an alternative cassette for an infusion pump, according to an embodiment of the invention.

FIG. 4C is an unassembled, perspective view of a reusable pressure plate adapter with an alternative cassette for an infusion pump, according to an embodiment of the invention.

FIG. 5 is a perspective view of a disposable cassette-type tube set for an infusion pump, according to an embodiment of the invention.

FIG. 6 is a front plan view of an assembled infusion pump with a reusable cassette and disposable tube set, according to an embodiment of the invention.

FIG. 7 is an unassembled perspective view of an infusion pump system which uses a reusable pressure plate arrangement with a disposable administration tube set, according to an embodiment of the invention.

FIG. 8A is an exploded view of a reusable pressure plate adapter for an infusion pump for an administration arrangement and various tube set components, according to an embodiment of the invention.

FIG. 8B is an exploded view of a reusable pressure plate adapter for an infusion pump for an administration arrangement and various tube set components, according to an embodiment of the invention.

FIG. 9 is a perspective view of a disposable administration type tube set for an infusion pump, according to an embodiment of the invention.

FIG. 10 is a front plan view of an assembled infusion pump with a reusable pressure plate and disposable administration tube set, according to an embodiment of the invention.

### DETAILED DESCRIPTION OF THE PREFERRED EMBODIMENTS

The invention may be embodied in other specific forms without departing from the essential attributes thereof. The illustrated embodiments should be considered in all respects as illustrative and not restrictive.

In various embodiments of this invention, apparatus and methods are disclosed for a pressure plate adapter system. In various embodiments, this device adapts the chassis of control modules of previous or current infusion pumps for use with primarily reusable components and generally require only basic disposable fluid carrying tube sets with specialized interface card components for operation. Accordingly, components of partially-reusable, partially-disposable systems are provided. The adapter for this type of device can generally be coupled against the chassis of a new or existing pump module and locked into place.

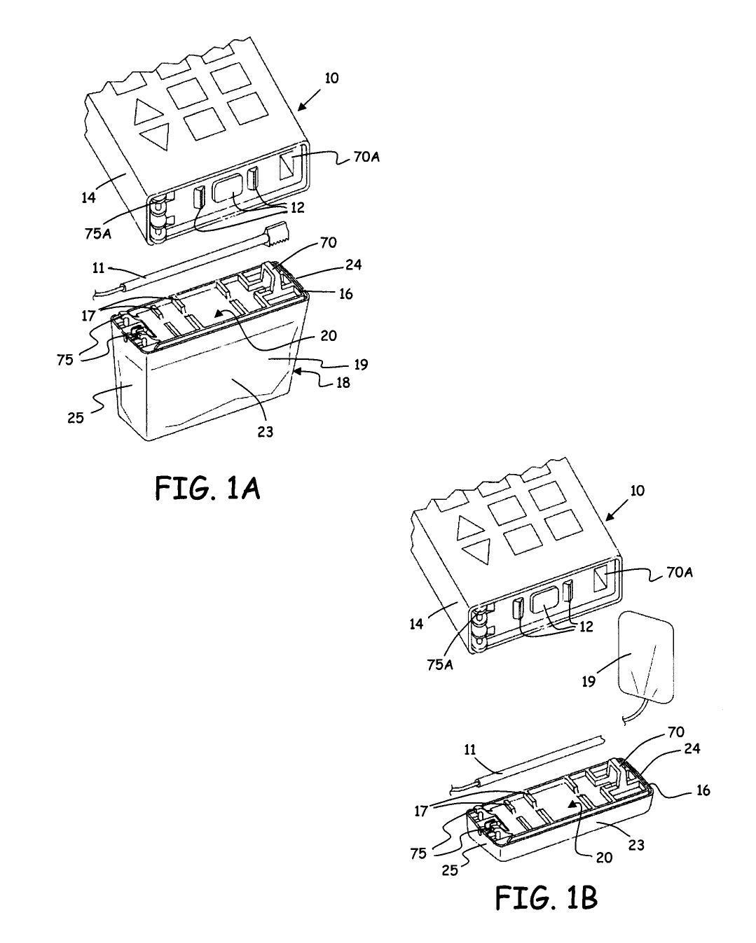

FIGS. 1A and 1B depict examples of illustrative prior art arrangements for infusion pumps. These and other types of arrangements for both reusable and disposable cassettes and pressure plates for infusion pumps can be further understood from various patents that disclose some common structural and/or operational characteristics. A wide variety of patents disclose some embodiments relating to infusion pump cassettes, base plates, and general operation, such as U.S. Pat. Nos. 4,565,542, 5,531,697, 5,531,698, 5,647,854, 5,658,252, 5,788,671, 5,823,746, 5,928,196, 5,954,485, 6,056,522, 6,059,753, and 6,123,686, for example, which are each hereby incorporated by reference in their entirety. The following brief explanation of the two types of prior arrangements shown in FIGS. 1A and 1B, will help better describe the differences and improvements provided by embodiments of the invention set forth in the remaining figures and description.

First, in FIG. 1A, a pump control module 10 is shown which is operative in effecting the movement of fluid through a tube 11. For the purposes of illustration, pump control module 10 includes a body 14 and valves 12. As illustrated, pump control module 10 is used with a cassette 18 having a pressure plate 20. Pressure plate 20 includes a top and bottom surface, two side walls 23 as well as an upstream wall 24 and a downstream wall 25. Cassette 18 includes a source of fluid contained within a bag 19 or a syringe (not shown). FIG. 1B shows a similar prior art arrangement, but where bag 19 or other fluid reservoir is positioned remote from pump control module 10 and pressure plate 20. In both arrangements, pressure plate 20 includes a plurality of guides 17 positioned on the top of pressure plate 20 which keep tube 11 in place on pressure plate 20. Pressure plate 20 also includes a rail 16 which surrounds pressure plate 20 and engages with body 14 of pump control module 10.

As illustrated in the prior art designs of FIGS. 1A and 1B, pressure plate 20 attaches to pump 10 with hooks 75 and loop 70. In particular, hooks 75 engage pins 75A of pump control module 10, and loop 70 is received within recess 70A. Recess 70A includes a latch (not shown) which selectively engages loop 70 to lock pressure plate 20 to pump control module 10. When pressure plate 20 is locked to pump control module 10, tube 11 is continually engaged by at least one of valves 12 such that tube 11 is constantly occluded. When in operation, valves 12 selectively squeeze tube 11 against pressure plate 20 to effect the movement of fluid through tube 11.

Applicants recognize that a number of advantages may be possible by improving the prior art designs of FIGS. 1A and 1B and related prior art designs. Specifically, while infusion pumps using cassette type tube arrangements have provided very convenient and reliable means for delivering medication, these cassettes are typically disposable and result in a fair amount of waste and added cost as a new cassette must be provided every time a new supply of medication is required. Further, filling sealed disposable cassettes and eliminating air bubbles, etc. from these cassettes can be time consuming and cause some difficulties to persons like pharmacy technicians responsible for filling these components.

Improvements to delivery accuracy and precision in administration-type arrangements and cassette-type arrangements are desired. This is because even small deviations in medications administered can potentially have significant effects on a user making accuracy and reliability of the upmost importance. Further, operation of components on the pressure plate is not always optimal and leaves considerable room for improvement.

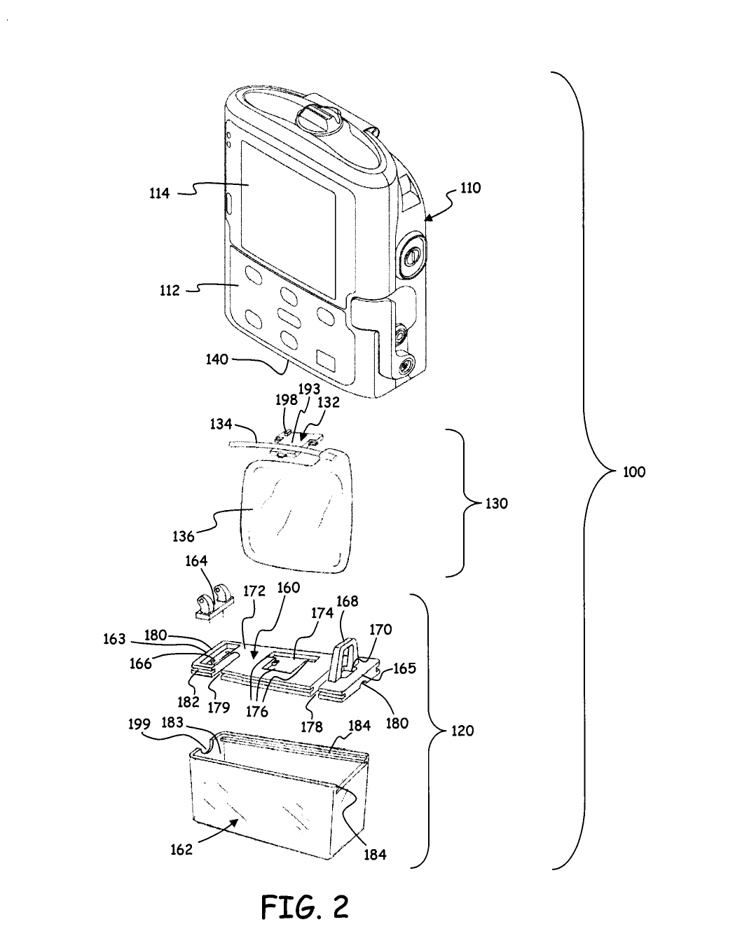

FIG. 2 illustrates an embodiment of a new unassembled infusion pump system 100 which includes an infusion pump module 110, a reusable cassette assembly 120, and a disposable cassette tube set 130. The disposable components of the cassette tube set 130 generally include an interface card component 132, a fluid tube 134, and a fluid cassette bag 136. More detailed views of embodiments of parts and configurations of this infusion pump system and cassette assembly are described throughout FIGS. 2-6.

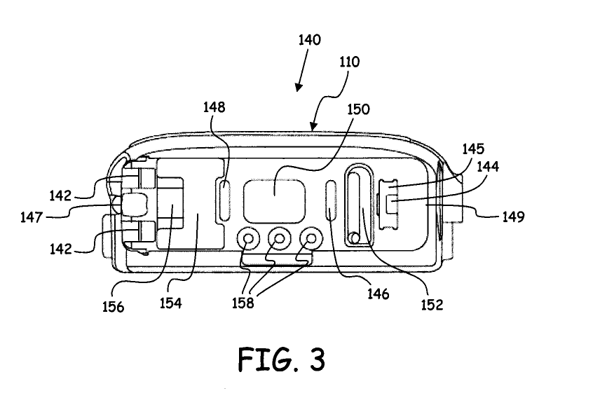

In general, the infusion pump control module 110 represents a general infusion pump or other similar type of pump. Infusion pumps are well-known and can provide a wide variety of fluid delivery protocols and capabilities. For example, the control module 110 may include a control system including a processor (not shown) electrically interconnected to a keypad 112, display 114, and pumping mechanism. The bottom of the control module 110 provides a pumping mechanism, among other components, and is responsible for the actual coordinated physical interaction and movement of components against the tubing of the tube set 130 to urge fluid through the fluid tube 134. This bottom region of the pump control module 110 is known as the chassis 140. The components of the chassis 140 can be better seen and described in the bottom view of the pump module shown in FIG. 3.

As depicted in FIG. 3, the lower surface of the control module 110 includes hinge pins 142 and a recess 145 containing a latching mechanism 144 at generally opposite ends 147 and 149 of the chassis 140. The hinge pins 142 and latching mechanism 144 are structures that can be used to join and lock the control module 110 and the reusable cassette assembly 120 together when the control module 110 and cassette assembly 120 are joined. The control module 110 further includes an inlet valve 146, an outlet valve 148 and a centrally located expulsor 150 to interact with an adjacent fluid tube 134 when the infusion pump is assembled. Also, as depicted in FIG. 3, the lower surface of the control module 110 includes an upstream occlusion sensor 152, a downstream occlusion sensor 154, an air detector sensor 156 and a plurality of detection pins 158. Theses sensors 152, 154, 156 and pins 158 help to ensure that the pump is able to monitor operation, ensure correct application of delivery protocols, provide alarms and recognize various characteristics of an adjacent tube set.

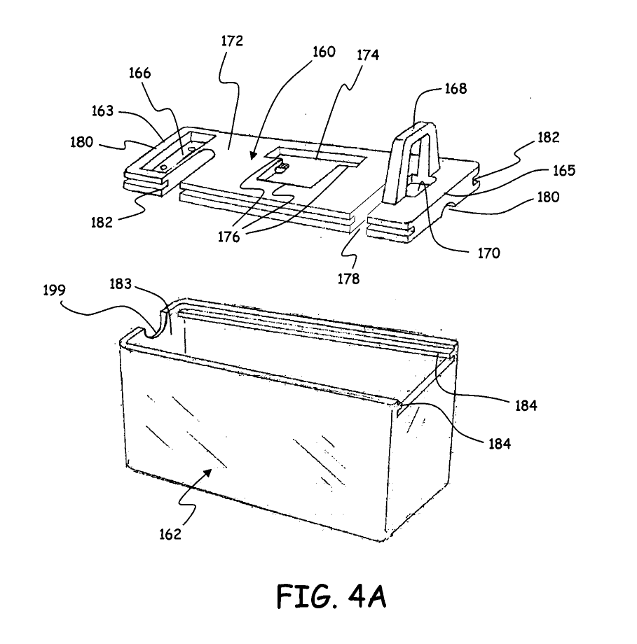

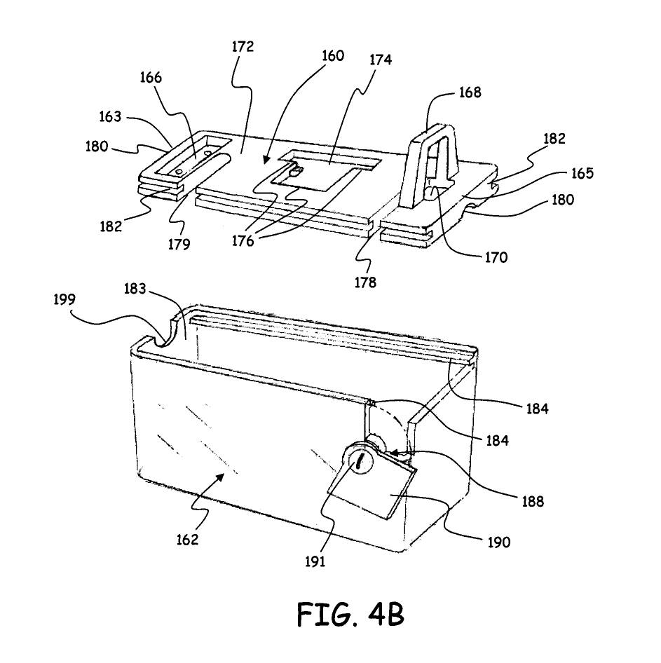

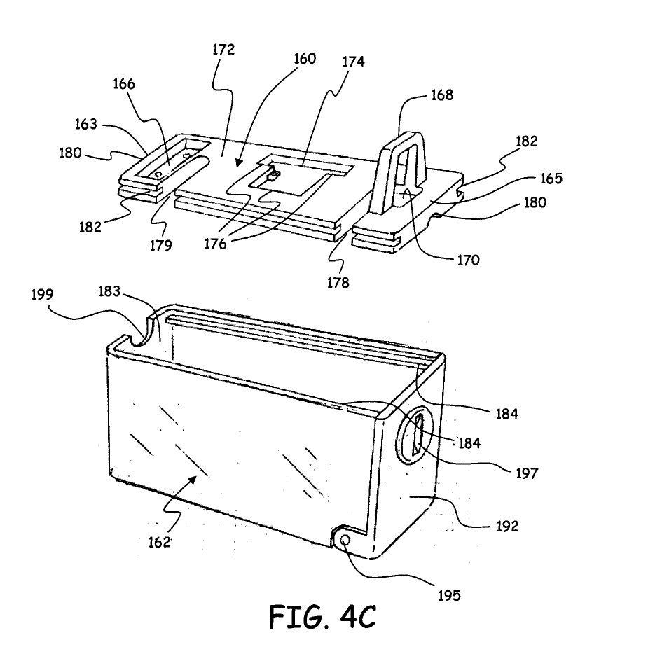

As shown in FIGS. 2, 4A, 4B and 4C, the reusable cassette assembly 120 includes an adapter 160 and reusable cassette housing 162 that are configured for slidable coupling with respect to one another. The adapter 160 is a moveable mechanism component that is capable of serving as a reusable replacement to pressure plate components of prior designs. The adapter 160 is generally an elongate body extending between a first end 163 and a second end 165. The adapter 160 remains coupled to the pump module 110, at least on one end, even when no cassette housing or cassette tube set 130 is attached to the control module 110. Accordingly, the first end 163 is pivotally mounted to the chassis of the control module for long-term pivotal retention, in the sense that the adapter is retained during repeated use over a period of time in which multiple tube sets and bags of fluid are utilized without the adapter 160 being separated or readily separable. Specifically, the retention arrangement is distinct from arrangements like the hook-based pressure plate arrangements of the prior art which are easily disengaged from hinge pins on the infusion pump chassis. The disclosed long-term retention arrangement has a permanent or semi-permanent pivot that cannot be quickly and easily separated by a user. Typically a user would need proper tools, or would need to undertake non-trivial retrofitting efforts to remove the adapter 160 from the module 110.

The adapter 160 is pivotally attached at one end 163 to the hinge pins 142 of the pump module chassis via a pivot mount 164 that is secured within a recess 166 in the top surface of the adapter 160. Coupling the pivotal mount 164 of the adapter 160 to the hinge pins 142 may be done to modify existing pumps already out in the field or to new pumps when manufactured. Accordingly, the adapter 160 provides the capabilities and advantages of a backwards compatible system. The adapter 160 is made of metal or other durable material that can withstand repeated use.

The second end 165 of the adapter 160, opposite the recess 166 and pivotal mount 164, contains a loop 168. The loop 168 is configured to fit within a recess 145 in the chassis 140 of the pump control module 110 and to be secured to a latching mechanism 144 when the adapter 160 is pivoted into a closed position adjacent the control module 110. Accordingly, the end 165 is configured for selective mounting to the chassis 140. In general, the loop 168 extends its arch shaped structure over a portion of the adapter 160 having an opening 170. This opening 170 provides access through the adapter 160 and is generally the location from which the fluid tube of an assembled pump emerges from a cassette housing 162, below the upper surface 172 of the adapter 160.

At the center of the adapter 160 is a T-shaped recess or opening 174. The T-shaped opening 174 is generally a recessed aperture in the upper surface 172 of the adapter 160 that enables an interface card component 132 associated with a cassette tube set 130 to be inserted and locked into place. The perimeter of the T-shaped opening 174 contains undercut portions 176 or related features which permit an interface card component 132 to be slid into a locked position. Opening 174 should not be understood as being limited to a T-shaped arrangement in various embodiments, but rather to include a variety of other possible shapes or configurations.

Further, one or more channels 178 and 179 may be located in the adapter 160 which permit necessary access and manipulation passageways for the fluid tube 134 when joining a reusable cassette assembly 120 and cassette tube set 130. Examples of such channels 178 and 179 are shown in FIGS. 4A-C which extends from the opening 170 under the loop 168 to the side of adapter 160. Fewer or additional channels of various shapes and configurations could be used as well to accommodate the fluid tube of the assembly when the infusion pump 100 is in the process of being assembled.

Also included on opposite ends of the adapter 160 are positive stop/capture components 180. These positive stop/capture components 180 allow easy alignment of the fluid tube 134 as well as correct fluid tube spacing. Specifically, the components 180 help to align the fluid tube, restrict fluid tube movement and retain portions of the fluid tube 134 so that the desired portion of the fluid tube 134 which is exposed to the expulsor and valves of the control module 110 is appropriately maintained.

The adapter 160 further includes a slide capture feature 182 located about the perimeter of the adapter. This slide capture feature 182 may be present on some or all the side surface of the adapter 160. Specifically the slide capture feature 182 is a groove shaped for slideably receiving and retaining a lip protrusion 184 of the reusable cassette housing 162. The lip protrusion 184 extends along two or more of the interior walls of the reusable cassette housing 162 just below the top perimeter of the housing 162.

In general, the reusable housing 162 is a multi-sided structure having an upwardly facing opening and open interior cavity 183. The housing 162 is used to house a fluid reservoir such as a flexible polymeric bag 136. The housing 162, shown in FIGS. 2, 4A, 4B, and 4C, is adapted for slidable engagement with the adapter 160. When engaging, the lip protrusions 184 of housing 162 are inserted into the slide capture features 182 of the adapter 160. The reusable cassette assembly 120 is not limited to this type of slidable coupling arrangement. Various other techniques of snaps of other coupling may be used to releasably join the adapter 160 and housing 162 and do not necessarily require a slidable arrangement. Further, the size of the cassette housing 162 is not limited and may be shaped to handle whichever size fluid bag 136 is deemed appropriate. Housings 162 of various sizes and shapes may be used to support and maintain fluid cassette bags 136 of various sizes and shapes.

FIGS. 4A, 4B, and 4C each show examples of different possible embodiments of the reusable cassette assembly 120. The cassette assembly in FIG. 4A is generally a more detailed view of the assembly 120 shown in FIG. 2 which relies upon a slideable arrangement. The cassette assembly in FIG. 4B is similar, but includes a lock mechanism 188. The lock mechanism 188 includes a lock feature 190 which can be rotated about a pivot member 191. In such an arrangement, once the cassette housing 162 is fully slid onto the adapter 160, an operator may rotate the lock feature 190 such that an impediment to further movement is provided. Accordingly, this lock feature 190 prevents further slideable movement of the cassette housing 162 with respect to the adapter 160. In such an arrangement, the cassette bag 136 is surrounded by a protective housing and may not be readily accessed or disturbed. FIG. 4C shows another cassette arrangement having a lockable door 192. The door 192 may be rotated open about a pivot 195 near the base of the cassette housing 162. The ability to open the door 192 allows for easy loading of a fluid bag 136 and manipulation of tubing 134. A lock 197 is present on the door 192 and can be used to restrict access to the cassette and secure the door 192 in place when closed. Locks 197 of various types and mechanisms may be used.

The cassette housing 162 further shows a tube notch 199 at the top of the wall opposite the door 192 to aid in the assembly of the cassette assembly 120 and tube set 130. This notch 199 provides an opening through which tubing can pass. A notch 199 of this type may be located in any of the cassette embodiments disclosed or described.

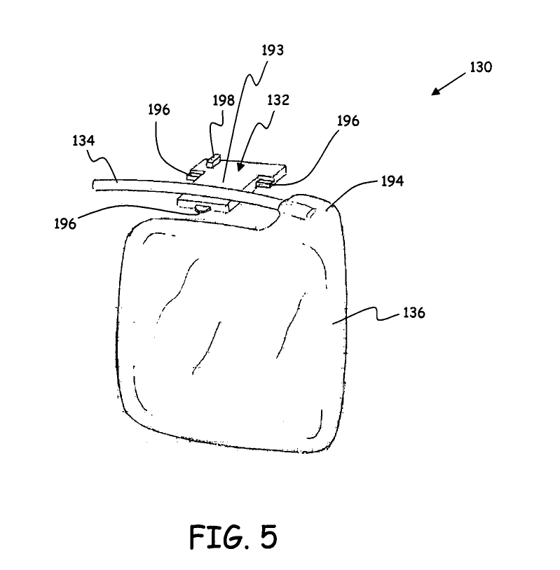

Further, a disposable tube set 130 is shown in FIG. 5 for use with a reuseable cassette assembly 120. The cassette tube set 130 includes an interface card component 132, fluid tube 134 and a fluid cassette bag 136. As these components largely represent the entire disposable structure of this infusion pump arrangement in some embodiments, much less waste is required than previous designs which included an entire cassette housing and/or pressure plate to be disposed of after use.

In general, the cassette bag 136 is a polymeric bag designed to house drugs, fluids or other medicants. The bag 136 may be various shapes and sizes and is not limited to the embodiment set forth in FIG. 5. The bag 136 is generally intended to be placed in the interior 183 of the reusable cassette housing 162. In some embodiments, the bag 136 includes an upper protruding portion 194 which can at least partially extend into the opening 170 of the adapter 160 below the loop 168 when the cassette assembly is assembled. In such an embodiment, the fluid tube 134 can conveniently extend outwardly from the side of upper protruding portion 194. The fluid tube 134 may be of various sizes depending upon the fluid or desired delivery protocol for the fluid. Tubes 134 may have various lengths, and thicknesses and may include additional filters or check valves in certain embodiments.

Further, fluid tube 134 is attached to an interface card component 132. This attachment may be accomplished using various processes or techniques. For example, in some embodiments these components are coupled using adhesive boding. In other embodiments, the components are coupled using a solvent bond with ring features on the interface card component 132. The interface card component 132 is generally coupled to an intermediate location of the tube 134, somewhere between the ends of the tube 134.

In certain embodiments, the interface card component 132 is a small, largely flat, stiff, generally T-shaped piece of material. The interface card component 132 may be made of any applicable material or materials, including plastics, metals, carbon-fibers, or reinforced polymers, for example. The interface card component 132 is not limited to a flat design and may be a component of various features protrusions, lips, edges, shapes, ribs, and engagement or locking features. The interface card component 132 is not limited to a T-shaped design and be designed in a variety of shapes of multi-sided, curved and/or linear sides. Circular, rectangular or I-shaped card components may be possible. The interface card component 132 typically has a generally flat top surface portion 193 for coupling to an exterior surface of the fluid tube 134. The interface card component 132 is used to provide a properly aligned interface for the fluid tube 134 to contact and interact with the chassis 140 of the control module 110. The interface card component 132 provides the correct spacing of the tube and cassette under the expulsor 150, which is often important to ensure delivery accuracy of the pump. In some embodiments, the interface card component 132 contains a ring feature that can be used to improve delivery accuracy by preventing tube expansion between the valves and expulsor.

The T-shaped interface card component 132 is configured to fit within the recess provided by the T-shaped opening 174 in the adapter 160. The interface card component 132 may include lower protrusions 196 to allow interlocking of the T-shaped interface card component 132 with the undercut portions 176 surrounding the T-shaped opening 174 in the adapter 160. The lower protrusions 196 may include outwardly extending segments of material of round or straight edges. In some embodiments, no lower protrusions 196 or locking features are present. Accordingly, the interface card component 132 is slightly smaller than the T-shaped opening 174, so that is may be readily aligned and inserted into the T-shaped opening, then slid into a locked configuration where the protrusions 196 of the interface card component 132 meet the undercut portions 176.

The interface card component 132 further includes a protrusion feature 198 on its upwardly facing surface. The protrusion feature 198 is used to interface with one or more of the detection pins 158 on the chassis of the control module 110. The chassis and/or the control module 110 could be altered in some embodiments to contain additional sensor locations, that could be activated by additional corresponding protrusion features 198 or other information conveying structure or component. In some embodiments, the protrusion feature 198 can relay information indicating the type of tube set and disposable being used, the tube size, or other relevant information about the disposable tube set 130. Use of detection pins to interact with such features to receive identifying information is well-known. Examples of patents discussing use of such components include U.S. Pat. Nos. 5,531,697, 5,531,698, and 6,123,686, hereby incorporated by reference in their entireties.

In general, the interface card component 132 is useful in many ways. It provides correct spacing under the expulsor 150 which dictates pump delivery accuracy. Further, the component 132 aligns the tubing 134 under the expulsor 150 to prevent “tube walk” or other undesired tube movement. The component 132 eliminates arch height effects of past designs, can prevent tube expansion between the valves and expulsor, and is able to use features that interface with detection pins 158 to indicate relevant information like the type of disposable being used.

When undergoing assembly, the fluid tube 134 of the cassette tube set 130 can be placed through openings or channels like opening 170 or channel 178 in order to pass through the adapter 160 and extend across the upper adapter surface 172 such that the interface card component is centrally located in the T-shaped opening 174.

Note that various flow stop valves or fluid flow protection devices may be used as part of embodiments of the present invention that may be adapted to conform to the components provided for this assembly.

Assembly of an infusion pump system according to embodiments of the invention will now be described. In an embodiment like the one shown in FIG. 2, the adapter 160 is first coupled to the chassis 140 of the control module 110. This may be done through via connection of the pivotal mount 164 between the adapter 160 and the hinge pins 142 of the chassis 140. The adapter 160 may be secured by the pivotal mount at the time of control module manufacture or after retrofit of existing pump modules present in the field by qualified personnel.

When a new cassette of medication is required for administration to a patient, a bag 136 associated with a cassette tube set 130 is filled by a pharmacist or other appropriate source and provided to the individual assembling the infusion pump assembly. The bag 136 is placed in the open interior 183 of the cassette housing 162. The ability to handle the bag 136 outside the confines of the cassette housing 162 is advantageous as the pharmacist or other individual can readily manipulate the bag and eliminate unwanted air bubbles and properly situate the fluid as desired. Next, the adapter 160 is coupled to the cassette housing 162. This may be done in an arrangement like the one shown in FIGS. 1-6 via sliding the lip protrusions 184 of the cassette housing 162 in the slide capture features 182 of the adapter 160. The fluid tube 134 associated by the filled bag 136 must be positioned and routed during this procedure to avoid interference with the adapter 160 as the adapter 160 is coupled to the cassette housing 162. The fluid tube 134 must be threaded through one or more of the channels 178, 179 or opening 170 at the appropriate time either after, during, or prior to coupling the adapter 160 with the cassette housing 162.

Next, the interface card component 132 is secured into the T-shaped opening 174 in the adapter and is locked into place. Next, the portions of the fluid tube 134 proximate each end of the adapter 160 are secured to positive stop/capture components 180. Finally, the reusable cassette assembly 120, together with the loaded cassette tube set 130, is rotated in a pivotal fashion about the hinge pins 142 that are engaged by the pivotal mount 164 until the upper surface 172 of the adapter 160 is adjacent the chassis 140 of the control module 110. When this occurs, the loop 168 is inserted into the recess 145 of the chassis 140 and is engaged by the latching mechanism 144 to hold the adapter 160 and remainder of the combined assembly in place adjacent the control module 110. A user may then initiate use of the control module 110 to deliver fluids or medicants via the infusion pump system 100.

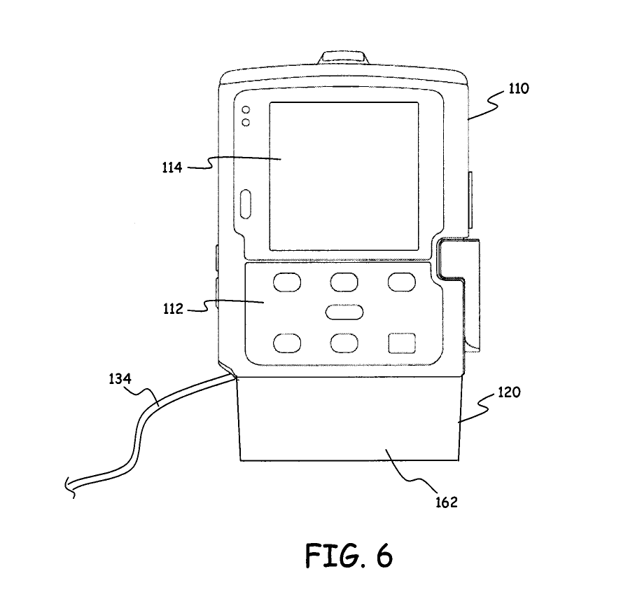

FIG. 6 depicts a front view of an infusion pump having a reusable cassette and disposable tube set, like the one of FIG. 2, but in an assembled state. The infusion pump control module 110, reusable cassette assembly 120 and fluid tube 134 for patient connection and delivery are depicted. For the most part, the adapter 160 cannot be seen due to its location below the control module 110 and beneath the top perimeter of the reusable cassette housing 162. Accordingly, the assembled infusion pump system 100 of various embodiments of the present invention outwardly appears much like the infusion pump designs of the prior art once assembled.

Various methods for a medical infusion pump pressure plate adapter system are made possible by this disclosure. One method includes providing a disposable tube set 130 adapted for attachment to a reusable adapter plate 160 having a reusable cassette reservoir housing 162. In this method, the tube set includes a fluid tube 134 with a bonded interface card component 132 and a fluid bag 136. The method further includes providing instructions to fill the bag of fluid 136, place the bag of fluid 136 into an interior portion 183 of the cassette reservoir housing 162 of the infusion pump, couple an interface card component 132 within a recess 174 of the reusable adapter plate, align the fluid tube 134 between a pair of positive stop components 180, and secure the reusable adapter plate 160 and the cassette reservoir housing 162 together by slideable engagement, hooks, tabs, or other securing means.

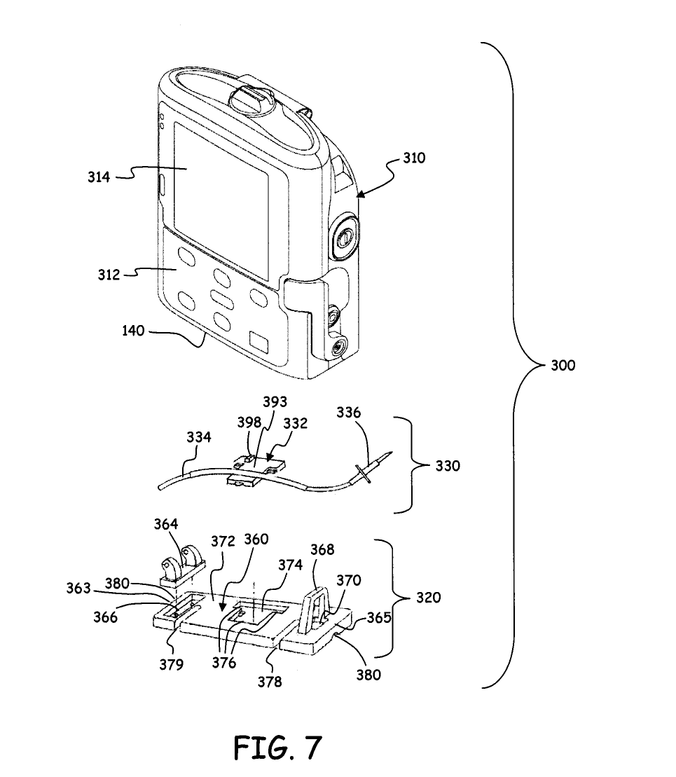

FIG. 7 illustrates an embodiment of an unassembled infusion pump system 300 which includes an infusion pump module 310, a reusable assembly 320, and a disposable administration tube set 330. The disposable components of the administration tube set 330 generally include an interface card component 332, a fluid tube 334, and a bag spike 336 for use with a remote medication reservoir or IV bag 337. Detailed views of various embodiments, configurations and aspects of this infusion pump system and cassette assembly are depicted in FIGS. 7-10. Although the embodiments depicted in FIGS. 7-10 are generally somewhat different from those shown in FIGS. 1-6, the descriptions of features and description previously set forth should be understood to be readily combined, incorporated, or applied to corresponding or similar embodiments and disclosures in FIGS. 7-10 and the related description.

In general, the infusion pump module 310 represents a general infusion pump or similar type of pump. Such pumps are well-known and can provide a wide variety of fluid delivery protocols and capabilities. For example, the control module 310 may include a control system including a processor (not shown) electrically interconnected to a keypad 312, display 314, and pumping mechanism. The bottom of the control module 310 provides a pumping mechanism, among other components, and is responsible for the actual coordinated physical interaction and movement of components against the tubing of the tube set 330 to urge fluid through the fluid tube 334. The bottom of the pump module 310 contains a chassis 140, as set forth in FIG. 3 and previously described above.

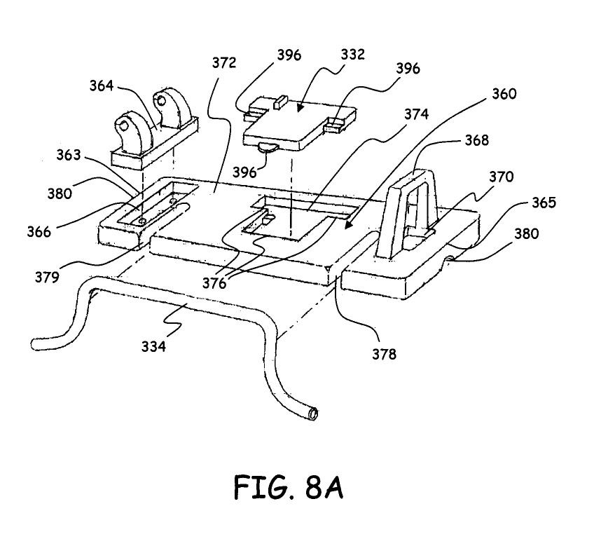

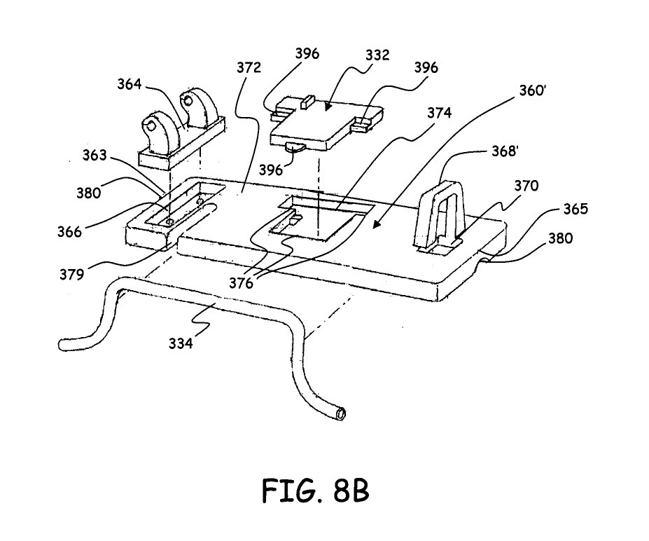

As shown in FIGS. 7, 8A and 8B, the reusable assembly 320 includes an adapter 360 configured for coupling with the control module 310. The adapter 360 is a moveable mechanism component that functions as a reusable replacement to pressure plate components of prior designs. Once assembled, the adapter 360 here is made to remain partially coupled to the pump module 310 at all times, even when no tube set 330 is attached to the infusion pump control module 310.

The adapter 360 is generally an elongate body extending between a first end 363 and a second end 365. The adapter 360 is made to remain coupled to the pump module 310, at least on one end, even when no cassette housing or cassette tube set 330 is attached to the control module 130. Accordingly, the first end 363 is pivotally mounted to the chassis of the control module for long-term pivotal retention, in the sense that the adapter is retained during repeated use over a period of time in which multiple administration tube sets of fluid are utilized without the adapter 360 being separated or readily separable from the control module 310. Specifically, the retention arrangement is distinct from arrangements like the hook-based pressure plate arrangements of the prior art which are easily disengaged from hinge pins on the infusion pump chassis. The disclosed long-term retention arrangement has a permanent or semi-permanent pivot that cannot be quickly and easily separated by a user. Typically a user would need proper tools or would need to undertake non-trivial retrofitting efforts to remove the adapter 360 from the module 310.

The adapter 360 is pivotally attached at one end to the hinge pins 142 of the pump module chassis 140 via a pivot mount 364 and may be secured within a recess 366 in the top surface of the adapter 360. Coupling the pivotal mount 364 of the adapter 360 to the hinge pins 142 may be done to modify existing pumps already out in the field or when new pumps when manufactured. Accordingly, the adapter 360 provides the capabilities and advantages of a backwards compatible system. The adapter 360 is made of metal or other durable material that can withstand repeated use.

The second end 365 of the adapter 360, opposite the recess 366 and pivotal mount 364, contains a loop 368. The loop 368 is configured to fit within a recess 145 in the chassis 140 of the pump control module 310 and to be secured to a latching mechanism 144. In general the loop 368 extends its arch shaped structure over a portion of the adapter having an opening 370. This opening 370 provides access through the adapter 360 and is generally the location from which the fluid tube 334 of an assembled pump emerges through to the upper surface 372 of the adapter 360.

At the center of the adapter 360 is a T-shaped opening 374. The T-shaped opening 374 is generally a recessed aperture enable an interface card component 332 associated with a administration tube set 330 to be inserted and locked into place. The perimeter of the T-shaped opening 374 contains undercut portions 376 or related features which permit an interface card component 332 to be slid into a locked position. Opening 174 should not be understood as being limited to a T-shaped arrangement in various embodiments, but rather to include a variety of other possible shapes or configurations

Further, one or more channels, such as the first channel 378 and second channel 379, depicted in FIG. 7, may be located in the adapter 360 which permit necessary access and manipulation passageways for the fluid tube of the administration tube set 330 when joining a reusable assembly 320 and administration tube set 330. Examples of an adapter 360 with two channels 378 and 379 are depicted in FIGS. 7 and 8A. FIG. 8B depicts an adapter 360′ with only a single channel 379. The channel 378 of these adapters extends from the opening 370 under the loop 368 to the side of the adapter 360. The channel 379 of these adapters extends from a midpoint of the adapter just inside the recess 366 to the side of the adapter 360. One or more other channels of various shapes and configurations could be used as well to accommodate the fluid tube of the assembly when the infusion pump 300 is in the process of being assembled.

Additionally included on opposite ends of the adapter 360 are positive stop/capture components 380. These positive stop/capture components 380 allow easy alignment of the fluid tube 334 as well as correct fluid tube spacing. Specifically, the components help to align the fluid tube, restrict fluid tube movement and retain portions of the fluid tube 334 so that the portion of the fluid tube exposed to the expulsor and valves of the control module 310 is appropriately maintained.

Although not depicted in FIG. 7, the adapter 360 may further include a slide capture feature (not shown) located about the perimeter of the component, as seen in FIGS. 2, 4A, 4B and 4C. Inclusion of such a slide capture feature may enable the adapter 160 to optionally function with multiple configurations. For example, the same adapter may function as part of an reusable cassette assembly 120 as discussed in FIGS. 2-6 or as a reusable assembly 320 set forth here.

FIGS. 8A and 8B each show examples of different possible embodiments of the adapters 360 and 360′ as well as interface card component 332 and fluid tube 334. These figures further include a pivotal mount 364. The adapter 360 in FIG. 8A is generally an elongated view of the adapter depicted in FIG. 7. The adapter 360′ in FIG. 8B is similar, but includes only one channel 379 and a loop 368′ with an incomplete structure that is only joined to the upper adapter surface 372 on one side. This partial structure allows for additional flexibility in tube movement when assembling the adapter 360. Such a partial structure could be used in any of the adapters discussed in this application as well as other adapter embodiments.

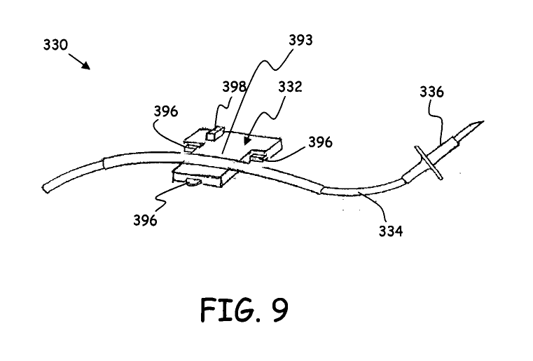

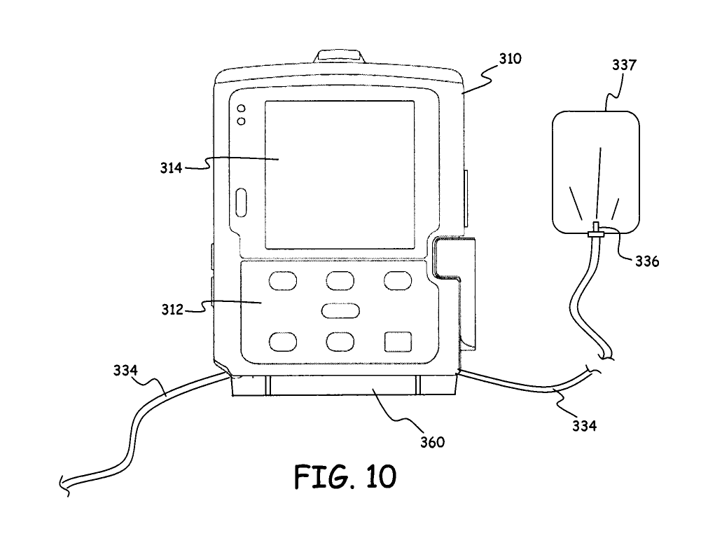

Further, a disposable administration tube set 330 is shown in FIG. 9 for use with a reusable assembly 320. The administration tube set 330 includes an interface card component 332, fluid tube 334 and a bag spike 336. As these components largely represent the entire disposable structure of this infusion pump arrangement, much less waste is required than previous designs which included disposable pressure plates. In certain embodiments no bag spike is included and another mechanism for remote reservoir attachment is used.

In general, the bag spike 336 may be used to pierce and open flow to a remote IV or fluid bag 337 housing drugs, fluids or other medicants. Further, fluid tube 334 is attached to an interface card component 332. In some embodiments, the tube 334 and interface card component 332 are coupled using adhesive bonding and in other embodiments, the components 332 and 334 are coupled using a solvent bond with ring features on the interface card component 332. The interface card component 332 is generally coupled to an intermediate location of the tube 334, somewhere between the ends of the tube 334.

In certain embodiments, the interface card component 332 is a small, largely flat, stiff, generally T-shaped piece of material. The interface card component 332 is not limited to a flat design and may be a component of various features protrusions, lips, edges, shapes, ribs, and engagement or locking features. The interface card component 332 typically has a generally flat top surface portion 393 for coupling to an exterior surface of the fluid tube 334.

Interface card component 332 is used to provide a properly aligned interface for the fluid tube 334 to contact and interact with the chassis 140 of the control module 310. The interface card component 332 provides the correct spacing under the expulsor 150, which is often important to ensure delivery accuracy of the pump. In some embodiments, the interface card component 332 contains a ring feature that can be used to improve delivery accuracy by preventing tube expansion between the valves and expulsor.

The T-shaped interface card component 332 is configured to fit within the recess provided by the T-shaped opening 374 in the adapter 360. The interface card component 332 may include lower protrusions 396 to allow interlocking of the T-shaped interface card component 332 with the undercut portions 376 surrounding the T-shaped opening 374 in the adapter 360. Accordingly, the interface card component 332 is slightly smaller than the T-shaped opening 374, so that it may be readily aligned and inserted into the T-shaped opening, then slid into a locked configuration where the protrusions 396 of the interface card component 332 meet the undercut portions 376.

The interface card component 332 further includes a protrusion feature 398 on its upwardly facing surface. The protrusion feature 398 is used to interface with the detection pins 158 on the chassis 140 of the control module 310. This can relay information indicating the type of disposable being used, tube size, or other relevant information about the disposable tube set 330.

When undergoing assembly, the fluid tube 334 of the administration tube set 330 can be placed through openings or channels like opening 370 or channels 378 or 379 in order to pass through the adapter 360 and extend across the upper adapter surface 372 such that the interface card component is centrally located in the T-shaped opening 374.

Assembly of this infusion pump system 300 will now be described. In an embodiment like the one shown in FIG. 7, the adapter 360 is first coupled to the chassis 140 of the control module 310. This may be done via connection of the pivotal mount 364 between the adapter 360 and the hinge pins 142 of the chassis 140. The adapter 360 may be secured by the pivotal mount 364 at the time of control module manufacture or after retrofit of existing pump modules present in the marketplace by qualified personnel.

When a new administration set is required for administration to a patient, the fluid tube 334 is threaded through the channels 378 and 379 or opening 370 of the adapter 360. Next, the interface card component 332 is secured into the T-shaped opening 374 in the adapter and is locked into place. Next, the portions of the fluid tube 334 proximate each end of the adapter 360 are secured to positive stop/capture components 380. Finally, the reusable assembly 320 is rotated in a pivotal fashion about the hinge pins 142 engaged by the pivotal mount 364 until the upper surface 372 of the adapter 360 is adjacent the chassis 140 of the control module 310. When this occurs, the loop 368 is inserted into the recess 145 of the chassis 140 and is engaged by the latching mechanism 144 to hold the adapter and combined assembly in place adjacent the control module 310. A user may then connect the bag spike 336 to a remote reservoir bag 337 and initiate use of the control module to deliver fluids or medicants via the infusion pump system 300.

FIG. 10 depicts a front view of an infusion pump having a reusable adapter 360 and a disposable tube set 330, like the one of FIG. 7, but in an assembled state. The infusion pump control module 310, reusable assembly 320 and fluid tube 334 for patient connection and delivery are depicted. For the most part, although the adapter 360 can be seen below the control module 310, the assembled infusion pump system outwardly appears much like the infusion pump designs for administration sets of the prior art once assembled.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the invention as set forth in the appended claims and the legal equivalents thereof.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A medical infusion pump pressure plate adapter system, comprising:
a tube set including:
a fluid tube for supplying fluid, medication, or nutrients; and
an interface card component having a generally flat top surface portion fixed to the exterior surface of the fluid tube at an intermediate location of the fluid tube;
a control module including:
a processor; and
a chassis with a pumping mechanism for pumping fluid through the fluid tube; and
a reusable adapter plate having a first end pivotally mounted to the chassis of the control module for long-term pivotal retention and a second end selectively, detachably mounted to the chassis of the control module, the reusable adapter plate containing a recessed feature configured for receipt of the interface card component of the tube set.

2. The pressure plate adapter system of claim 1, wherein the tube set includes a fluid bag coupled to the fluid tube.

3. The pressure plate adapter system of claim 1, wherein the tube set includes a bag spike coupled to the fluid tube.

4. The pressure plate adapter system of claim 1, wherein the interface card component is T-shaped.

5. The pressure plate adapter system of claim 1, wherein the interface card component is coupled to the fluid tube by adhesive bonding.

6. The pressure plate adapter system of claim 1, wherein the interface card component is coupled to the fluid tube by solvent bonding between a ring feature on the interface card component and the fluid tube.

7. The pressure plate adapter system of claim 1, wherein the interface card component contains a raised protrusion for interfacing with one or more cassette detection pins on the chassis.

8. The pressure plate adapter system of claim 1, wherein the interface card component is adapted to communicate the type of tube set being used.

9. The pressure plate adapter system of claim 1, wherein the interface card component is sized to dictate the spacing for the fluid tube relative to an expulsor on the chassis.

10. The pressure plate adapter system of claim 1, wherein the reusable adapter plate contains a plurality of positive stops to align the fluid tube and restrict fluid tube movement.

11. The pressure plate adapter system of claim 1, wherein the reusable adapter plate contains channels to route the fluid tube.

12. The pressure plate adapter system of claim 1, wherein the reusable adapter plate is made of metal.

13. The pressure plate adapter system of claim 1, wherein the reusable adapter plate contains a slide feature about the perimeter that is configured to engage a reusable cassette housing.

14. The pressure plate adapter system of claim 1, wherein the reusable adapter plate is selectively coupled to a cassette.

15. The pressure plate adapter system of claim 14, wherein the cassette includes a lock for restricting access to an interior portion of the cassette.

16. A disposable tube set for a medical infusion pump, comprising:
a fluid tube for supplying fluids, medication, or nutrients; and
an interface card component with a generally flat top surface portion coupled to the exterior surface of the fluid tube at an intermediate location of the fluid tube, the interface card component configured to releasably couple to a recessed feature of a reusable adapter plate of an infusion pump.

17. The disposable tube set of claim 16, wherein the tube set includes a fluid bag.

18. The disposable tube set of claim 16, wherein the tube set includes a bag spike.

19. The disposable tube set of claim 16, wherein the interface card component is T-shaped.

20. The disposable tube set of claim 16, wherein the interface card component is coupled to the fluid tube by adhesive bonding.

21. The disposable tube set of claim 16, wherein the interface card component is coupled to the fluid tube by solvent bonding between a ring feature on the interface card component and the fluid tube.

22. The disposable tube set of claim 16, wherein the interface card component contains a raised protrusion for interfacing with one or more cassette detection pins on a medical infusion pump.

23. The disposable tube set of claim 16, wherein the interface card component is adapted to communicate the type of tube set being used.

24. The disposable tube set of claim 16, wherein the interface card component is sized to dictate the spacing for the fluid tube relative to an expulsor of a medical infusion pump.

25. A reusable adapter plate for use with a control module of an infusion pump, comprising:
an elongate body having a first end with a pivotal mounting structure with one or more apertures for long-term pivotal infusion pump attachment to the control module, a second end including a pump securing extension for selective engagement with the control module, and a top portion configured to retain an interface card component that is coupled to a fluid tube between the first end and the second end.

26. The pressure plate adapter system of claim 25, wherein the reusable adapter plate contains a plurality of positive stops to restrict fluid tube movement.

27. The pressure plate adapter system of claim 25, wherein the reusable adapter plate contains one or more access channels.

28. The pressure plate adapter system of claim 25, wherein the reusable adapter plate is made of metal.

29. The pressure plate adapter system of claim 25, wherein the reusable adapter plate contains a slide feature about the perimeter that is configured to engage a cassette housing.

30. The pressure plate adapter system of claim 25, wherein the reusable adapter plate is coupled to a cassette.

31. The pressure plate adapter system of claim 30, wherein the cassette includes a lock for restricting access to an interior portion of the cassette.

32. A method for a medical infusion pump pressure plate adapter system, comprising:
providing a disposable tube set adapted for attachment to a reusable adapter plate having a reusable cassette reservoir housing, the tube set including a fluid tube with a bonded interface card component and a fluid bag; and
providing instructions to:
fill the bag of fluid;
place the bag of fluid into an interior portion of the cassette reservoir housing of the infusion pump;
couple an interface card component within a recess of the reusable adapter plate and align the fluid tube between a pair of positive stop components; and
secure the reusable adapter plate and the cassette reservoir housing together.
